(19) United States
(12) Patent Application Publication
(10) Pub. No.: US 2004/0204489 A1
(43) Pub. Date: Oct. 14, 2004

(54) Method of treating a swollen gland curing cancer

(76) Inventor: Arthur Vanmoor, Bocaraton, FL (US)

Correspondence Address: Otto S. Kauder, 5382 C Venetia Court, Boynton Beach, FL, 33437, US

(21) Appl. No.: 10/414,140

(22) Filed: Apr. 14, 2003

(51) Int. Cl. A61K 31/198; A61K 31/195

(52) U.S. Cl. 514/563; 514/564

(57) ABSTRACT

There is disclosed a method of treating a swollen gland in a person in need of such treatment, which comprises the administration to such person of an effective amount of at least one compound represented by formula (I)

X—CH₂—CH(OY)₁₋ₚ(H)ₚCH(NH₂⁺)ₚCOO⁻ (I)

wherein X represents a trimethylammonio group (CH3)3N+ or a carbamoyl group (CONH₂), Y is hydrogen or an acetyl (CH₃CO) group, and p is zero or one, provided that when X is a carbamoyl group p is one, and when X is a trimethylammonio group p is zero.

### BACKGROUND OF THE INVENTION

1. Field of the Invention

This invention relates to a method of treating a person suffering from a swelling of a gland and associated discomfort with an agent effective in counteracting that condition and minimizing associated pain and discomfort.

2. Description of the Related Art

Glands are specialized organs of the human body that secrete liquid substances within or outside the body. Glands that secrete liquid substances within the body are known as endocrine glands and their secretions are known as hormones. Endocrine glands include, for example, the thyroid gland, the pituitary gland, the adrenal gland, the pancreas, as well as the gonads, i.e. the female ovaries and the male testes. Glands secreting substances that exit the body include the liver, the gall bladder, the kidney, the salivary gland, and, located in or on the body's exterior, i.e. the skin, for example the sweat glands, and the sebaceous glands

In a healthy individual, the glands perform their function unnoticed, without being seen or felt. A number of disease conditions, however, including many forms of cancer, are accompanied by swelling or increase in size of one or more glands, possibly as a result of the body's efforts to combat the disease and rid itself of foreign substances such as toxins and irritants. The victim can actually see the increase in size of a gland located on or within the skin, and may feel pain as a result of a swollen gland within the body exerting pressure on adjacent organs and nerves.

As is well known, the search by conventional methods for better remedies for gland abnormalities such as swelling and associated discomfort and pain is enormously costly. For economic reasons, moreover, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

In contrast, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds are disclosed in U.S. Pat. No. 5,616,617 as effective against facial pimples; certain water soluble amino carboxylic acid compounds are disclosed in U.S. Pat. No. 5,626,831 as effective against the common cold; certain water soluble amino carboxylic acid compounds are disclosed in U.S. Pat. No. 5,707,967 as effective against headache; certain water soluble amino carboxylic acid compounds are disclosed in U.S. Pat. No. 5,708,029 as effective against elevated blood pressure, and certain water soluble amino carboxylic acid compounds are disclosed in U.S. Pat. No. 5,767,157 as effective against pain in a joint.

For evident reasons, however, this method is not applicable to the study and relief of cancerous conditions.

There clearly remains a need for new approaches to finding and applying more effective and also less aggressive techniques for diminishing and preferably eliminating swelling of a gland in a non-aggressive manner.

### SUMMARY OF THE INVENTION

In accordance with this invention, there is provided a method of treating a swollen gland in a person in need of such treatment, which comprises the administration to such person of an effective amount of at least one compound represented by formula (I) shown below, effective in diminishing the size of the gland and preferably returning the gland to its normal size.

X—CH₂—CH(OY)₁₋ₚ(H)ₚCH(NH₂⁺)ₚCOO⁻ (I)

wherein X represents a trimethylammonio group (CH3)3N+ or a carbamoyl group (CONH₂), Y is hydrogen or an acetyl (CH₃CO) group, and p is zero or one, provided that when X is a carbamoyl group p is one, and when X is a trimethylammonio group p is zero.

When the swelling of the gland accompanies a cancer condition, treating the swelling in accordance with the invention can put the cancer in remission and possibly cure it.

The effectiveness of the compound represented by formula (I) according to the invention is believed to accompany enhancement of the effectiveness of the person's immune system. Enhancing the effectiveness of the immune system in a person can be beneficial in augmenting the person's innate ability to eliminate toxins from the body and to resist the initiation of the process that leads to a gland being swollen, as well as to slow down, arrest, and even reverse that process. As a result, the incidence of pain and other discomfort associated with such swelling is diminished, and the quality of life is improved.

In minimizing swelling of a gland according to this invention, mega-nutrient doses of 2 to 20 grams of at least one compound represented by formula (I) can be administered from one to five times daily, for a total of 2 to 100 grams per day, until monitoring shows sufficient improvement in the user's condition to permit reduction in dose level and ultimately cessation of the treatment. Daily doses of a compound represented by formula (I) in the range from 2 to 40 grams per day are preferred. Such doses can be administered in any convenient manner, as by oral administration in any of the usual dosage forms, such as tablets, capsules, solutions, and dispersions in liquid foods such as soups and fruit juices. Alternatively, there can be given sterile solutions of a compound represented by formula (I) by direct injection into the bloodstream of the person to be treated, as well as by rectal suppositories containing a compound represented by formula (I).

When the gland is located on the surface of the body, such as a sweat gland or a sebaceous gland, the swelling and the effect of treatment on the swelling is readily monitored by inspection. For monitoring swelling of glands internal to the body, and the results of treatment according to the invention, well known techniques including the administration of contrast media that make the gland visible by fluoroscope or x-ray are available.

In formula (I), when X is a carbamoyl group the compound is that represented by formula II

H2NCO—CH₂—CH₂₋CH(NH₂⁺)COO⁻ (II)

When X is a trimethylammonio group the compound can be that represented by formula (III)

(CH3)3N+-CH₂—CH(OH)CH₂COO⁻ (III)

as well as its acetylated derivative represented by formula (IV)

(CH3)3N+-CH₂—CH(OCOCH3)CH₂COO⁻ (IV)

### EXAMPLE 1

A male volunteer noticed a swelling in his crotch area that grew from a one-eighth inch bump when first noticed to a half inch after five days. He then took five grams total of a combination of compounds II, III, and IV daily for five days and after that time the swelling had totally disappeared.

### EXAMPLE 2

A female volunteer noticed a swelling in her left armpit area that grew from a quarter-inch bump when first noticed to a half inch after seven days. She then took ten grams total of compounds II, III, and IV daily for seven days and after that time the swelling had diminished to about half the size before beginning treatment. She then reduced the daily dose to six grams for one week after which time the swelling had disappeared

What is claimed is:

1. A method of treating a person suffering from a swelling of a gland which comprises the administration to such person of an effective amount of at least one compound represented by formula (I)
X—CH₂—CH(OY)₁₋ₚ(H)ₚCH(NH₂⁺)ₚCOO³¹ (I)
wherein X represents a trimethylammonio group (CH3)3N+ or a carbamoyl group (CONH₂), Y is hydrogen or an acetyl (CH₃CO) group, and p is zero or one, provided that when X is a carbamoyl group p is one, and when X is a trimethylammonio group p is zero.

2. The method of claim 1, wherein the compound is administered orally with food.

3. The method of claim 1, wherein the compound is administered by injection into the bloodstream.

4. The method of claim 1, wherein the compound is administered by rectal suppository.

5. The method of claim 1, wherein the effective amount of the compound is administered in one to five daily doses, each dose being in the range of 2 to 20 gram.

6. The method of claim 1, wherein the total of said effective amount of the compound administered daily is in the range of 2 to 100 grams.

7. The method of claim 6, wherein the total of said effective amount of the compound administered daily is in the range of 20 to 50 grams.

8. The method of claim 1, wherein said person experiences reduction in the size of the swelling.

9. The method of claim 1 wherein after treatment the size of the gland has returned to normal.

10. The method of claim 1 wherein the compound is
H2NCO—CH₂—CH₂₋CH(NH₂⁺)COO⁻

11. The method of claim 1 wherein the compound is
(CH3)3N+-CH₂—CH(OH)CH₂COO⁻

12. The method of claim 1 wherein the compound is
(CH3)3N+-CH₂—CH(OCOCH3)CH₂COO⁻

13. The method of claim 1, wherein a plurality of compounds represented by formula (I) is administered.

14. The method of claim 1 wherein the gland is a sweat gland.

15. The method of claim 1, wherein the gland is a sebaceous gland.

16. The method of claim 1, wherein the gland is an endocrine gland.

17. The method of claim 1, wherein the gland is selected from the group consisting of the liver, the gall bladder, the kidney, and the salivary gland.

18. A method of treating a person suffering from a swelling of a gland which comprises enhancing the person's immune system by the administration to such person of an effective amount of at least one compound represented by formula (I)
X—CH₂—CH(OY)₁₋ₚ(H)ₚCH(NH₂⁺)ₚCOO⁻ (I)
wherein X represents a trimethylammonio group (CH3)3N+ or a carbamoyl group (CONH₂), Y is hydrogen or an acetyl (CH₃CO) group, and p is zero or one, provided that when X is a carbamoyl group p is one, and when X is a trimethylammonio group p is zero.

19. The method of claim 18, wherein the gland is a sweat gland.

20. The method of claim 18, wherein the gland is a sebaceous gland.
